(19) United States
(12) Patent Application Publication
(10) Pub. No.: US 2006/0115906 A1
(43) Pub. Date: Jun. 1, 2006

(54) Sensor surface comprising an improved signal-noise ratio

(76) Inventor: Holger Klapproth, Freiburg (DE)

Correspondence Address: GRIFFIN & SZIPL, PC, SUITE PH-1, 2300 NINTH STREET, SOUTH, ARLINGTON, VA, 22204, US

(21) Appl. No.: 10/521,817

(22) PCT Filed: Jul. 21, 2003
(86) PCT No.: PCT/DE03/02452

§ 371 Date: Sep. 7, 2005

(30) Foreign Application Priority Data
Jul. 22, 2002 (DE) 10233303.3

(51) Int. Cl. G01N 33/543

(52) U.S. Cl. 436/518

(57) ABSTRACT

The present invention concerns a sensor surface with an improved signal/noise ratio that is based on a blocking reagent covalently immobilized on the sensor surface, as well as apparatuses comprising such surfaces and methods for the use of same.

The present invention concerns a sensor surface with an improved signal/noise ratio that is based on a blocking reagent covalently immobilized on the sensor surface, as well as apparatuses comprising such surfaces, and methods for the use of same.

For sensors that operate on the receptor/ligand principle, for example antibody/antigen sensors (protein sensors), most often none of the reaction partners or only the receptor is covalently immobilized. After the immobilization (application) of the receptor, usually a blocking reagent or agent is added prior to the actual reaction in order to hinder nonspecific binding, that is binding that does not take place exclusively at the receptor, of the analyte molecules on the sensor surface. This blocking reagent occupies unused regions on the sensor, and is mandatory for nearly all measurements in order to obtain a sufficiently high signal/noise ratio.

A problem that appears constantly in conventional sensors up to the present is that the blocking reagent can be displaced over the course of later reaction and washing steps. Loss of the reagent leads to (partial) exposure of surface regions on the sensor on which nonspecific interactions can again take place. Such an interaction or reaction would generate a nonspecific signal during detection, which would cause a significant deterioration in the ratio of specific signals to nonspecific signals.

The object of the present invention is to eliminate this disadvantage which is inherent in the conventional sensors of the prior art.

This problem is solved by the present invention through the provision of a sensor surface with covalently-immobilized, specific probe molecules for at least one biomolecule to be detected, where, in principle, the positions on or regions of the sensor surface that are available for nonspecific binding are inactivated by at least one blocking reagent covalently immobilized thereon.

In summary, the affinity sensors for biomolecules of the present invention that operate on the receptor/ligand principle are treated with a reagent following application of the receptor molecules, such that nonspecific interactions of the analyte molecules other than specific binding in the intended regions of said receptor(s) of the sensor surface will be eliminated through the covalent bonding of blocking reagents or blocking agents. In this way the signal/noise ratio for the reaction will be improved and the sensitivity of the analysis will be increased. Covalent immobilization or binding of the receptors and the blocking reagents to the sensor surface makes increased specificity of the reaction possible, for example, by employing higher surfactant concentrations, while hindering washout of the blocking reagents during the various washing steps that take place in order to remove unbound analyte from the sensor.

Since the signal/noise ratio with regard to the receptors is improved (this refers only to the biological signal/noise ratio and not to the electronic signal/noise ratio), the absolute sensitivity of the measurements is thus increased, and the sensor sensitivity is thereby improved as well as the dynamic range of the measurement being largely increased. In particular, the use of lipophilic photocrosslinkers and amphiphilic blocking reagents yields the possibility of selectively altering the surface characteristics without the risk of damaging or otherwise impairing the valuable receptor proteins. In this way, antibody chips can optionally be reused, since the structure and loading of the chip is maintained essentially intact.

Another aspect of the present invention relates to a method for improving the signal/noise ratio of a biosensor through the use of a sensor surface of the present invention. Thus the present invention relates in principle to any method for determining the presence of an analyte in a test sample to be analyzed through the use of surface-bound receptor molecules in which a sensor surface of the present invention is utilized.

An additional aspect of the present invention relates to an apparatus for use in a method of the present invention that possesses a sensor surface of the present invention.

A further aspect of the present invention relates to a kit for use in a method of the present invention, which contains a sensor surface of the present invention and optionally buffers and assay reagents.

Another aspect of the present invention relates to a blocking reagent that possesses at least one photoreactive group for covalent immobilization to a sensor surface.

An additional aspect of the present invention relates to a method for the production of a blocking reagent of the present invention, wherein at least one blocking reagent as defined above is reacted with at least one crosslinker that possesses at least one photoreactive group.

A further aspect of the present invention relates to a kit for producing a sensor surface of the present invention that contains at least one blocking reagent of the type defined previously, and optionally contains a sensor surface as well as buffers and reagents.

Additional advantageous and/or preferred embodiments of the present invention are the object of the respective subclaims.

In one embodiment of the sensor surface of the present invention, the probe or receptor molecules form an addressable pattern on the surface. Such patterns are known in principle from bioarray or biochip technology, and can be created by means of any of the techniques employed in those fields, for example imprinting. Array technology permits a very large number of analytes to be examined in parallel.

Any techniques can be used for immobilizing the probe molecules, for example those described by G. T. Hermanson in “Bioconjugate Techniques”, Academic Press, 1996. By way of example, in the case of amino-terminated oligonucleotides, so-called reactive esters such as N-hydroxysuccinimide (NHS-esters), epoxides, preferably glycidyl derivatives, isothiocyanates, isocyanates, azides, carboxylic acid groups or maleimides are suitable. Naturally, immobilization could also be effected by using the same photocrosslinkers, which will be described further below for the immobilization of the blocking reagents.

In another embodiment of the sensor surface of the present invention, the covalent immobilization of the at least one blocking reagent is effected by means of at least one photoreactive crosslinker. It is possible to use many different blocking reagents in parallel. In principle, any blocking agent known to the art is suitable for use in the present invention. Any blocking reagent can optionally be immobilized by a number of photoreactive crosslinkers which might also be different.

In a further embodiment of the sensor surface of the present invention, a minimum of one photoreactive crosslinker possesses at least one photoreactive group selected from among benzophenone or derivatives thereof, anthraquinone or derivatives thereof, thymidine or derivatives thereof, and 4-azidobenzoic acid or derivatives thereof. In principle, any photoreactive crosslinker known to the art is suitable for use in the present invention.

In an additional embodiment of the sensor surface of the present invention, the sensor surface is selected from among a metal, semimetal, semimetal oxide, glass or polymer surface.

In a further embodiment of the sensor surface of the present invention, the metal surface is selected from among gold and aluminum surfaces.

In another preferred embodiment of the sensor surface of the present invention, the semimetal surface is a silicon surface.

In an additional embodiment of the sensor surface of the present invention, the semimetal oxide surface is a silicon oxide or an aluminum oxide surface.

In a further embodiment of the sensor surface of the present invention, the glass surface is a quartz glass surface. In principle, any known glass surface is suitable for use in the present invention.

In principle, all surface shapes are suitable for use in the present invention. Although the surface in biochip applications is usually essentially flat, it is obvious to anyone skilled in the art that surfaces with wells as well as those that are not flat but are formed in a rounded or spherical shape are likewise suitable for use in the present invention.

In another embodiment of the sensor surface of the present invention, the polymer surface is selected from among surfaces of a cyclolefin copolymer or derivatives thereof, polystyrene or derivatives thereof, polyethylene or derivatives thereof, polypropylene or derivatives thereof, polyimide or derivatives thereof, and poly(methyl methacrylate) or derivatives thereof. In principle, any known polymer surface is suitable for use in the present invention. Thus the present invention also comprises surfaces that possess swellable or water-permeable polymeric or copolymeric structures, and can include mono-, bi- or polyfunctionalized coupling groups.

Furthermore, membranes known in the art for use in analysis and diagnosis, such as especially those of nylon and nitrocellulose, are suitable.

In an additional embodiment of the sensor surface of the present invention, the probe molecule (receptor) is a partner in a specific interaction system of complementary binding partners (receptor/ligand).

In a further embodiment of the sensor surface of the present invention, the specific interaction system of complementary binding partners is based upon an interaction between a nucleic acid with a complementary nucleic acid, an interaction of a peptide nucleic acid with a nucleic acid, an enzyme/substrate interaction, a receptor/effector interaction, a lectin/sugar interaction, an antibody/antigen interaction, an avidin/biotin interaction, or a streptavidin/biotin interaction.

In another embodiment of the sensor surface of the present invention, the nucleic acid is a DNA or RNA or an analog thereof.

In an additional embodiment of the sensor surface of the present invention, the DNA or RNA is an oligonucleotide.

In a further embodiment of the sensor surface of the present invention, the antibody is a polyclonal, monoclonal, chimeric, or single-chain antibody, or a functional fragment or derivative of such an antibody. Functional fragment or derivative of an antibody here is understood to be any fragment or derivative of an antibody with specific antigen-binding capability. The potency of this can be different from that of the native antibody, but this doesn't necessarily mean that the fragment or derivative at the same time also possesses an immunizing effect in the body.

In another embodiment of the sensor surface of the present invention, the blocking reagent is selected from among casein, hydrolyzed casein, a surfactant, bovine serum albumin, fetal calf serum, newborn calf serum, and mixtures thereof. These blocking reagents are available commercially, and for example may be purchased from Sigma-Aldrich Chemicals GmbH.

In an additional embodiment of the sensor surface of the present invention, the surfactant is selected from among sodium palmitate, Brij® 35, Brij® 58, cetylpyridinium chloride monohydrate, cetyltrimethylammonium bromide, 3-[(3-cholamidopropyl)dimethylammonio]-1-propanesulfonate, 3-[(3-cholamidopropyl)dimethylammonio]-2-hydroxy-1-propanesulfonate, decane-1-sulfonic acid sodium salt, N,N-bis-[3-(D-gluconamido)propyl]deoxycholamide, dodecane-1-sulfonic acid sodium salt, dodecyl-β-D-maltoside, 6-O—(N-heptylcarbamoyl)methyl-α-D-glucopyranoside, heptane-1-sulfonic acid sodium salt, N-lauroylsarcosine sodium salt, octanoyl-N-methylglucamide, N-nonaoyl-N-methylglucamide, sodium cholate, sodium deoxycholate, nonane-1-sulfonic acid sodium salt, Nonidet P40, octane-1-sulfonic acid sodium salt, n-octyl-β-D-glucopyranoside, pentane-1-sulfonic acid sodium salt, n-octyl-β-D-thioglupyranoside, Pluronic® F-68, saccharose monolaurate, sodium dodecyl sulfate, N-dodecyl-dimethyl-3-ammonio-1-propanesulfonate, N-tetradecyl-dimethyl-3-ammonio-1-propanesulfonate, Triton® X-100, and mixtures thereof. These surfactants are available commercially, and for example may be purchased from Sigma-Aldrich Chemicals GmbH. In principle, all known surfactants are suitable for use in the present invention.

Further details of the abovementioned surfactants are provided in the following table.

| Name | Empirical formula | Type |
|---|---|---|
| Brij ® 35 | C₅₈H₁₁₈O₂₄ | nonionic |
| Brij ® 58 | C₅₆H₁₁₄O₂₁ | nonionic |
| Cetylpyridinium chloride | C₂₁H₃₈CIN × H₂O | cationic |
| monohydrate | | |
| Cetyltrimethylammonium | C₁₉H₄₂BrN | cationic |
| bromide | | |
| CHAPS | C₃₂H₅₈N₂O₇S | zwitterionic |
| CHAPSO | C₃₂H₅₈N₂O₈S | zwitterionic |
| Decane-1-sulfonic acid sodium | C₁₀H₂₁NaO₃S | |
| salt | | |
| Deoxy-BIGCHAP | C₄₂H₇₅N₃O₁₆ | nonionic |
| Dodecane-1-sulfonic acid | C₁₂H₃₅NaO₃S | |
| sodium salt | | |
| Dodecyl-β-D-maltoside | C₁₂H₃₅NaO₃S | nonionic |
| HECAMEG | C₁₅H₂₉NO₇ | nonionic |
| Heptane-1-sulfonic acid sodium | C₇H₁₅NaO₃S × H₂O | |
| salt | | |
| N-Lauroylsarcosine sodium salt | C₁₅H₂₈NNaO₃ | anionic |
| MEGA-8 | C₁₅H₃₁NO₆ | nonionic |
| MEGA-9 | C₁₆H₃₃NO₆ | nonionic |
| Sodium cholate | C₂₄H₃₉NaO₅ | anionic |
| Sodium deoxycholate | C₂₄H₃₉NaO₄ | anionic |
| Nonane-1-sulfonic acid sodium | C₉H₁₉NaO₃S | |
| salt | | |
| Nonidet P40 | Mixture of 15 homologs | |
| Octane-1-sulfonic acid sodium | C₈H₁₇NaO₃S | |
| salt | | |
| n-Octyl-β-D-glucopyranoside | C₁₄H₂₈O₆ | nonionic |
| Pentane-1-sulfonic acid sodium | C₅H₁₁NaO₃S | |
| salt | | |
| n-Octyl-β-D- | C₁₄H₂₈O₅S | |
| thioglucopyranoside | | |
| Pluronic ® F-68 | n.a. | nonionic |
| Saccharose monolaurate | C₂₄H₄₄O₁₂ | nonionic |
| SDS (sodium dodecyl sulfate) | C₂₄H₄₄O₁₂ | anionic |
| Sulfobetaine SB 12 | C₁₇H₃₇NO₃S | zwitterionic |
| Sulfobetaine SB 14 | C₁₉H₄₁NO₃S | zwitterionic |
| Triton ® X-100 | | nonionic |
| Triton ® X-114 | C₃₀H₅₄O₉ | noniomc |
| Tween ® 20 | | nonionic |
| Tween ® 80 | | nonionic |

Abbreviations: CHAPS=3-(3-cholamidopropyl)dimethylammonio-1-propanesulfonate; CHAPSO=3-(3-cholamidopropyl)dimethylammonio-2-hydroxy-1-propanesulfonate; Deoxy-BIGCHAP=N,N-bis-[3-(D-gluconamido)propyl]deoxycholamide; HECAMEG=6-O—(N-heptylcarbamoyl)methyl-α-D-glucopyranoside; MEGA-8=octanoyl-N-methylglucamide; MEGA-9=N-nonanoyl-N-methylglucamide; SDS=sodium dodecyl sulfate; Sulfobetain SB 12=N-dodecyl-dimethyl-3-ammonio-1-propanesulfonate; Sulfobetain SB 14=N-tetradecyl-dimethyl-3-ammonio-1-propanesulfonate.

In a further embodiment of the blocking reagent of the present invention, the minimum of one photoreactive group is selected from among benzophenone or derivatives thereof, anthraquinone or derivatives thereof, thymidine or derivatives thereof, and 4-azidobenzoic acid or derivatives thereof.

In one embodiment of the method of the present invention for the production of blocking reagents of the present invention, the at least one photoreactive group is selected from among benzophenone or derivatives thereof, anthraquinone or derivatives thereof, thymidine or derivatives thereof, and 4-azidobenzoic acid or derivatives thereof.

The invention will be clarified in the following examples.

A suitable blocking reagent, for example casein for polystyrene surfaces, is provided with a photoreactive group by means of a crosslinker, for example 4-azidobenzoic acid N-hydroxysuccinimide ester. These chemical groups make it possible for the blocking reagents to be covalently immobilized onto the positions available to them following the actual blocking reaction. The surface can also optionally be provided with photoreactive groups, for example a glass surface that has been coated with a silane-benzophenone. Native blocking reagents can be used in this case, and the immobilization of the receptors and the blocking reagent takes place under exposure to light of a suitable wavelength.

Other blocking reagents suitable for the present invention are the abovementioned surfactants with a photoreactive group on the hydrophobic end, for example sodium benzophenone-4-palmitate. Derivatives, also of other surfactants, can be readily prepared by one skilled in the art. These substances will block regions in which hydrophobic interactions take place. Nonionic and anionic surfactants with photoreactive groups are also suitable. It would be an advantage also to be able to prepare stable lipid vesicles in this way.

### EXAMPLES

Preparation of Photoreactive Casein Fragments

It is preferable to use relatively low-molecular casein with a molecular weight<10 kD. This material is dissolved in sodium phosphate buffer (pH 7.5) and is reacted in the dark with 20 molar equivalents of a crosslinker (5-azido-2-nitrobenzoic acid N-hydroxysuccinimide ester as a stock solution in dimethylformamide (DMF)) (at room temperature for 2 h). Next, the protein is purified by separation on a Sephadex column and the concentration is adjusted to 1% (w/v), for example by dilution. The pH of the prepared solution is adjusted to 7.0.

Application to a Surface

The covalent blocking of the biosensor optionally provided beforehand with recptor molecules is carried out in the following manner. The sensor surface is incubated with the blocking buffer for 2 h at 4° C., and is then washed exhaustively with PBS buffer (a phosphate-buffered saline solution). Finally the preparation is exposed to UV light (wavelength ca. 300 inn) for ca. 5 min. Optionally, the sensor surface can also be wetted or sprayed with a 0.1% gelatin solution for better stabilization of the proteins that are present.

1-59. (canceled)

60. A sensor surface with covalently-immobilized specific probe molecules for at least one biomolecule to be detected, wherein positions on or regions of the sensor surface that are available in principle for nonspecific binding are inactivated by at least one blocking reagent covalently immobilized thereon, obtainable by a method in which the sensor surface is covered with the blocking reagent after a covalent immobilization of the probe molecule, and the blocking reagent comprises at least one photoreactive crosslinker having at least one photoreactive group suitable for covalent immobilization onto the sensor surface by irradiation.

61. The sensor surface as recited in claim 60, wherein said probe molecules form an addressable pattern.

62. The sensor surface as recited claim 60, wherein said photoreactive group is selected from the group consisting of benzophenone and derivatives thereof, anthraquinone and derivatives thereof, thymidine and derivatives thereof, and 4-azidobenzoic acid and derivatives thereof.

63. A sensor surface as recited in claim 60, wherein the sensor surface is selected from the group consisting of metal, semimetal, semimetal oxide, glass, and polymer surface.

64. The sensor surface as recited in claim 63, wherein the metal surface is selected from the group consisting of gold and aluminum surfaces.

65. The sensor surface as recited in claim 63, wherein the semimetal surface is a silicon surface.

66. The sensor surface as recited in claim 63, wherein the semimetal oxide surface is a silicon oxide or aluminum oxide surface.

67. The sensor surface as recited in claim 63, wherein the glass surface is a quartz glass surface.

68. The sensor surface as recited in claim 63, wherein the polymer surface is selected from the group consisting of surfaces of a cyclolefin copolymer and derivatives thereof, polystyrene and derivatives thereof, polyethylene and derivatives thereof, polypropylene and derivatives thereof, polyimide and derivatives thereof, and poly(methyl methacrylate) and derivatives thereof.

69. A sensor surface as recited in claim 60, wherein the probe molecule is a partner in a specific receptoraigand interaction system.

70. The sensor surface as recited in claim 69, wherein said specific receptor/ligand interaction system is selected from the group consisting of an interaction between a nucleic acid with a complementary nucleic acid, an interaction of a peptide nucleic acid with a nucleic acid, an enzyme/substrate interaction, a receptor/effector interaction, a lectin/sugar interaction, an antibody/antigen interaction, an avidin/biotin interaction, and-a streptavidin/biotin interaction.

71. The sensor surface as recited in claim 70, wherein the nucleic acid is selected from the group consisting of DNA, RNA, and analogs thereof.

72. The sensor surface as recited in claim 71, wherein the DNA or RNA is an oligonucleotide.

73. The sensor surface as recited in claim 70, wherein said antibody is selected from the group consisting of polyclonal, monoclonal, chimeric, and single-chain antibodies, and functional fragments and derivatives of such antibodies.

74. A sensor surface as recited in claim 60, wherein the blocking reagent is selected from the group consisting of casein, hydrolyzed casein, a surfactant, bovine serum albumin, fetal calf serum, newborn calf serum, and mixtures thereof.

75. The sensor surface as recited in claim 74, wherein said surfactant is selected from the group consisting of sodium palmitate, Brij® 35, Brij® 58, cetylpyridinium chloride monohydrate, cetyltrimethylammonium bromide, 3-[(3-cholamidopropyl)dimethylammonio]-1-propanesulfonate, 3-[(3-cholamidopropyl)dimethylammonio]-2-hydroxy-1-propanesulfonate, decane-1-sulfonic acid sodium salt, N,N-bis-[3-(D-gluconamido)propyl]deoxycholamide, dodecane-1-sulfonic acid sodium salt, dodecyl-β-D-maltoside, 6-O—(N-heptylcarbamoyl)methyl-a-D-glucopyranoside, heptane-1-sulfonic acid sodium salt, N-lauroylsarcosine sodium salt, octanoyl-N-methylglucamide, N-nonaoyl-N-methylglucamide, sodium cholate, sodium deoxycholate, nonane-1-sulfonic acid sodium salt, Nonidet P40, octane-1-sulfonic acid sodium salt, n-octyl-β-D-glucopyranoside, pentane-1-sulfonic acid sodium salt, n-octyl-β-D-thioglucopyranoside, Pluronic® F-68, saccharose monolaurate, sodium dodecyl sulfate, N-dodecyl-dimethyl-3-ammonio-1-propanesulfonate, N-tetradecyl-dimethyl-3-ammonio-1-propanesulfonate, Triton® X-100, and mixtures thereof.

76. A method for detecting the presence of analytes in a sample to be tested through the use of surface-bound receptor molecules, wherein a sensor surface as recited in claim 60 is used.

77. An apparatus for detecting the presence of analytes in a sample to be tested through the use of surface-bound receptor molecules, wherein the apparatus possesses a sensor surface as recited in claim 60.

78. A kit for detecting the presence of analytes in a sample to be tested through the use of surface-bound receptor molecules, wherein the kit contains a sensor surface as recited in claim 60 and optionally contains buffers and assay reagents.

79. A blocking reagent, wherein the blocking reagent has at least one photoreactive group for covalent immobilization on a sensor surface.

80. A blocking reagent as recited in claim 79, wherein the blocking reagent is selected from the group consisting of casein, hydrolyzed casein, a surfactant, bovine serum albumin, fetal calf serum, newborn calf serum, and mixtures thereof.

81. A blocking reagent as recited in claim 80, wherein the surfactant is selected from the group consisting of sodium palmitate, Brij® 35, Brij® 58, cetylpyridinium chloride monohydrate, cetyltrimethylammonium bromide, 3-[(3-cholamidopropyl)dimethylammonio]-1-propanesulfonate, 3-[(3-cholamidopropyl)dimethylammonio]-2-hydroxy-1-propanesulfonate, decane-1-sulfonic acid sodium salt, N,N-bis-[3-(D-gluconamido)propyl]deoxycholamide, dodecane-1-sulfonic acid sodium salt, dodecyl-β-D-maltoside, 6-O—(N-heptylcarbamoyl)methyl-α-D-glucopyranoside, heptane-1-sulfonic acid sodium salt, N-lauroylsarcosine sodium salt, octanoyl-N-methylglucamide, N-nonaoyl-N-methylglucamide, sodium cholate, sodium deoxycholate, nonane-1-sulfonic acid sodium salt, Nonidet P40, octane-1-sulfonic acid sodium salt, n-octyl-β-D-glucopyranoside, pentane-1-sulfonic acid sodium salt, n-octyl-β-D-thioglucopyranoside, Pluronic® F-68, saccharose monolaurate, sodium dodecyl sulfate, N-dodecyl-dimethyl-3-ammonio-1-propanesulfonate, N-tetradecyl-dimethyl-3-ammonio-1-propanesulfonate, Triton® X-100, and mixtures thereof.

82. A blocking reagent as recited in claim 79, wherein said minimum of one photoreactive group is selected from among benzophenone and derivatives thereof, anthraquinone and derivatives thereof, thymidine and derivatives thereof, and 4-azidobenzoic acid and derivatives thereof.

83. A method for the production of a blocking reagent, wherein at least one blocking reagent as recited in claim 79 is reacted with at least one crosslinker that possesses at least one photoreactive group.

84. The method as recited in claim 83, wherein the minimum of one photoreactive group is selected from among benzophenone or derivatives thereof, anthraquinone or derivatives thereof, thymidine or derivatives thereof, and 4-azidobenzoic acid or derivatives thereof.

85. A kit for the production of a sensor surface, wherein
the sensor surface has covalently-immobilized specific probe molecules for at least one biomolecule to be detected, wherein positions on or regions of the sensor surface that are available in principle for nonspecific binding are inactivated by at least one blocking reagent covalently immobilized thereon, obtainable by a method in which the sensor surface is covered with the blocking reagent after a covalent immobilization of the probe molecule, and the blocking reagent comprises at least one photoreactive crosslinker having at least one photoreactive group suitable for covalent immobilization onto the sensor surface by irradiation; and
wherein the kit contains the least one blocking reagent covalently immobilized on the sensor surface by means of at least one photoreactive group, and, optionally, buffers and assay reagents.

86. A method for producing a sensor surface, comprising the following steps:
(a) covalent immobilization of the probe molecules on the sensor surface;
(b) covalent immobilization of the blocking reagent comprising at least one photoreactive crosslinker with at least one photoreactive group onto the sensor surface by irradiation to produce a sensor surface with covalently-immobilized specific probe molecules for at least one biomolecule to be detected, wherein positions on or regions of the sensor surface that are available in principle for nonspecific binding are inactivated by at least one blocking reagent covalently immobilized thereon.

87. A method as recited claim 86, wherein said photoreactive group is selected from the group consisting of benzophenone and derivatives thereof, anthraquinone and derivatives thereof, thymidine and derivatives thereof, and 4-azidobenzoic acid and derivatives thereof.

88. A method according to claim 86, wherein the sensor surface is selected from the group consisting of metal, semimetal, semimetal oxide, glass, and polymer surface.

89. A method according to claim 86, wherein said probe molecule is a partner in a specific receptor/ligand interaction system.

90. A method according to claim 86, wherein said blocking reagent is selected from the group consisting of casein, hydrolyzed casein, a surfactant, bovine serum albumin, fetal calf serum, newborn calf serum, and mixtures thereof.

91. A method according to claim 90, wherein said surfactant is selected from the group consisting of sodium palmitate, Brij® 35, Brij® 58, cetylpyridinium chloride monohydrate, cetyltrimethylammonium bromide, 3-[(3-cholamidopropyl)dimethylammonio]-1-propanesulfonate, 3-[(3-cholamidopropyl)dimethylammonio]-2-hydroxy-1-propanesulfonate, decane-1-sulfonic acid sodium salt, N,N-bis-[3-(D-gluconamido)propyl]deoxycholamide, dodecane-1-sulfonic acid sodium salt, dodecyl-β-D-maltoside, 6-O—(N-heptylcarbamoyl)methyl-α-D-glucopyranoside, heptane-1-sulfonic acid sodium salt, N-lauroylsarcosine sodium salt, octanoyl-N-methylglucamide, N-nonaoyl-N-methylglucamide, sodium cholate, sodium deoxycholate, nonane-1-sulfonic acid sodium salt, Nonidet P40, octane-1-sulfonic acid sodium salt, n-octyl-β-D-glucopyranoside, pentane-1-sulfonic acid sodium salt, n-octyl-β-D-thioglucopyranoside, Pluronic® F-68, saccharose monolaurate, sodium dodecyl sulfate, N-dodecyl-dimethyl-3-ammonio-1-propanesulfonate, N-tetradecyl-dimethyl-3-ammonio-1-propanesulfonate, Triton® X-100, and mixtures thereof.
